(19) United States
(12) Patent Application Publication
(10) Pub. No.: US 2020/0155184 A1
(43) Pub. Date: May 21, 2020

(54) GRASPERS FOR USE IN GUIDING DELIVERY OF PERCUTANEOUS CARDIAC THERAPEUTIC DEVICES

(71) Applicant: Synecor LLC, Chapel Hill, NC (US)

(72) Inventors: Richard S. Stack, Chapel Hill, NC (US); William L. Athas, Chapel Hill, NC (US); Kevin Johnson, Durham, NC (US); Emer M. Feerick, Galway (IE); Matthew Moran, Galway (IE); Piotr Brzozowski, Galway (IE)

(21) Appl. No.: 16/578,380

(22) Filed: Sep. 22, 2019

Related U.S. Application Data
(60) Provisional application No. 62/802,212, filed on Feb. 7, 2019.
(60) Provisional application No. 62/744,689, filed on Oct. 12, 2018.
(60) Provisional application No. 62/744,692, filed on Oct. 12, 2018.
(60) Provisional application No. 62/744,696, filed on Oct. 12, 2018.

(51) Int. Cl. A61B 17/29; A61M 1/12

(52) U.S. Cl. CPC A61B 17/29; A61B 2017/2926; A61B 2017/2905; A61M 1/122

(57) ABSTRACT

A system for use in transseptally delivering a cardiac therapeutic device (“CTD”) to a target site in the heart, includes a CTD having an elongate flexible advancer on its distal end, and a flexible member proportioned for introduction into a vasculature and having a length to extend from the right subclavian vein, through the heart via a transseptal puncture, and to a femoral artery such that a first end of the flexible member is external to the patient at the right subclavian vein or other venous access site and a second end of the flexible member is external to the patient at the femoral artery. The flexible member has a grasper releasably engageable with the advancer.

This application claims the benefit of the following U.S. Provisional Applications: 62/744,689, filed 12 Oct. 2018, 62/744,692, filed 12 Oct. 2018, 62/744,696, filed 12 Oct. 2018, and 62/802.212, filed 7 Feb. 2019. Each of the above applications is incorporated herein by reference.

### BACKGROUND

Several new types of percutaneous interventional devices have recently been introduced that do not rely on a traditional guidewire for delivery to the heart. These non-guidewire based (NGB) devices include percutaneous ventricular assist devices as well as certain transvascular aortic or mitral valve repair or replacement devices. Percutaneous ventricular assist devices (pVADs) are pump devices positioned within the heart and used for circulatory support. In order for these pVADs to be considered minimally invasive, interventional cardiology-based procedures, they must enter the heart from a percutaneous puncture of a peripheral vessel. If the devices are thin and flexible, they may be introduced in an artery and advanced retrograde across the aortic valve to the left ventricle. If they are too large to enter an artery, they may be introduced into larger peripheral veins but then they must cross from the right side of the heart to the left side across the inter-atrial septum in a well-established but tortuous route via a technique known as transseptal catheterization. However, because of the combined large size and/or rigidity of these high cardiac output pVADS, generally the transseptal route has proven to be extremely difficult. One major obstacle is that pVAD designs generally involve a pump housing on the distal end which would prohibit passage of a traditional guidewire lumen through the housing.

The traditional transseptal approach involves driving a therapeutic device over a 0.035 in. guidewire that has been previously introduced across the interatrial septum, through the left atrium then across the mitral valve and into the left ventricle. This guidewire produces a highly flexible track over which these large devices can potentially be forced into position. However, high cardiac output pVADs are too big and too rigid to easily negotiate the tight bends that are required when crossing into and navigating through the left atrium, left ventricle and the aorta. As a result, they can fail to follow the course of the guidewire and continue in a relatively straight course when attempting to negotiate the multiple turns required, causing both the deformed guidewire and tip of the therapeutic device to protrude into the delicate cardiac tissues.

U.S. application Ser. No. 16/578,375, entitled Systems and Methods for Transseptal Delivery of Percutaneous Ventricular Assist Devices and Other Non-Guidewire Based Transvascular Therapeutic Devices, filed Sep. 22, 2019 and incorporated herein by reference (Attorney Ref: SYNC-5000R) discloses a system and method for delivering therapeutic devices positionable in the heart, particularly at the aortic valve, and particularly pVAD devices as well as other NGB devices, to the heart, together with exemplary methods for using those systems using superior venous access. It describes a system of components that allow the user to both push the proximal end of the pVAD while simultaneously pulling on the distal nose of it with equal and coordinated force to drive the pVAD through the vasculature and across the interatrial septum. During the course of a procedure using that system, an engagement device is used to engage the distal tip of the pVAD device. Before this engagement is carried out, the distal end of the engagement device is positioned so that its proximal end and handle extend out of the body from the left or right femoral artery, and its distal end extends out of the right subclavian vein (“RSV”), with the shaft between the proximal and distal ends extending between the arterial and venous side via the interatrial septum. Outside the RSV, the distal end of the engagement device is securely engaged to the tip of the pVAD device. This allows the pVAD to be moved through the vasculature and across the interatrial septum using a combination of pulling (by pulling on the engagement device from the left or right femoral artery) and pushing (by pushing on the proximal end of the pVAD from the RSV.

To carry out this step, engagement devices are needed that can securely couple to the pVAD's distal tip so that the coupled devices can be advanced together through the heart and/or vasculature. Engagement must be sufficiently secure to prevent release of the advancer from the pVAD despite the application of tension to the grasper from outside the femoral artery. For convenience, these engagement devices will be referred to as “graspers” and their function with respect to the pVAD may be referred to as “grasping.” The terms “grasper” and “grasping” are often associated with devices having jaw members, and this application shows and describes some grasper devices that include jaw members, one or both of which articulate to engage the feature on the pVAD that is to be engaged. However, it should be understood that the term grasper as used herein encompasses various other suitable configurations, some which do not have jaw members.

### BRIEF DESCRIPTION OF THE DRAWINGS

FIG. 1A illustrates a percutaneous ventricular assist device including an example of an advancer that may be engaged using grasper devices of the type described herein;

FIG. 1B is a side elevation view of the advancer shown in FIG. 1A;

FIG. 2 is a perspective view of a first embodiment of a grasper;

FIG. 3A is a perspective view of a distal end of the grasper of FIG. 3A and the tip of the advancer of FIG. 1B;

FIG. 3B is similar to FIG. 3A but shows the grasper engaged to the advancer;

FIG. 3C is a top plan view of the configuration of FIG. 3B, with the jaw members and advancer shown in cross-section;

FIG. 3D is a side elevation view of the configuration of FIG. 3B, with the jaw members and advancer shown in cross-section;

FIGS. 4A-4D are a sequence of drawings illustrating use of the grasper of FIGS. 2-3D to engage an advancer;

FIG. 5A is a perspective view of the handle of the grasper of FIG. 2;

FIG. 5B is a perspective view of an alternative to the handle of FIG. 5A;

FIG. 6A is a perspective view of the distal end of a second embodiment of a grasper;

FIG. 6B is similar to FIG. 6A but shows the advancer inserted between the jaws and inserted into the seat of the fixed jaw member prior to closing of the jaws;

FIG. 7A is a perspective view of a third embodiment of a grasper spaced apart from an advancer to be engaged;

FIG. 7B shows the grasper of FIG. 7A engaged to the advancer;

FIG. 7C is a top plan view of the advancer of FIG. 7A;

FIG. 7D is a perspective view of the grasper and advancer of FIG. 7A, showing the step of moving the advancer between the jaws of the jaw assembly in the method of engaging the advancer and the gasper;

FIG. 8 is a side elevation view of a fourth embodiment of a grasper, together with an advancer;

FIGS. 9A-9D are a sequence of drawings illustrating use of a fifth embodiment of a grasper to engage a pVAD device;

FIGS. 10A and 10B are a sequence of drawings illustrating use of a sixth embodiment of a grasper to engage a pVAD device;

FIG. 10C is a perspective view of the snare catheter of FIG. 10A;

FIG. 10D is similar to FIG. 10C, but shows the catheter without the snare;

FIG. 11A is a perspective view of a seventh embodiment of a grasper;

FIGS. 11B-11D are a sequence of drawings illustrating use of the seventh embodiment to engage a pVAD device;

FIGS. 12A and 12B are a sequence of drawings illustrating use of an eighth embodiment to engage an advancer of a pVAD device;

FIG. 12C is a close-up view of the distal end of the grasper of FIGS. 12A and 12B;

FIGS. 13A-13D are a sequence of drawings illustrating use of a ninth embodiment to engage an advancer of a pVAD device;

FIG. 14A is a partially exploded perspective view showing a tenth embodiment of a grasper. The grasper is shown with the cap in a separated position to permit its contents to be seen;

FIG. 14B shows the grasper of FIG. 14B engaging an advancer;

FIG. 14C shows the grasper of FIG. 14B releasing the advancer. The cap is not shown in FIG. 14C, for clarity.

FIGS. 15A and 15C are side elevation views, rotated 90 degrees from one another, showing an eleventh embodiment of a grasper engaged with an advancer.

FIGS. 15C and 15D are similar to FIG. 15A but show the steps in which the advancer is being released from the grasper.

FIGS. 16A and 16C are side elevation views, rotated 90 degrees from one another, showing a twelfth embodiment of a grasper engaged with an advancer.

FIGS. 16C and 16D are similar to FIG. 16A but show the steps in which the advancer is being released from the grasper.

FIGS. 17A and 17B are side elevation views, rotated 90 degrees from one another, showing a thirteenth embodiment of a grasper engaged with an advancer.

FIGS. 17C and 17D are similar to FIG. 17A but show the steps in which the advancer is being released from the grasper.

FIG. 17E is a side elevation view of a slighted modified version of the thirteenth embodiment, using an alternate shape of the nitinol element to provide a ledge for the snare loop to engage.

FIGS. 18A and 18B are perspective views of a distal part of a fourteenth embodiment of a grasper, with the collet in the compressed and expanded positions, respectively.

FIG. 19A is a cross-sectional side elevation view of a distal part of a fifteenth embodiment of a grasper.

FIGS. 19B-19E are a sequence of drawings illustrating use of the grasper of FIG. 19A to engage and release and advancer.

FIG. 20A is a cross-sectional side elevation view of a sixteenth embodiment of a grasper inserted into a lumen of an advancer;

FIG. 20B is a side-elevation view similar to FIG. 20A, showing the out member advanced over the advancer. The advancer is shown transparent.

FIGS. 20C and 20D are a sequence of drawings illustrating the steps of detaching the grasper from the disk that is within the advancer.

### DETAILED DESCRIPTION

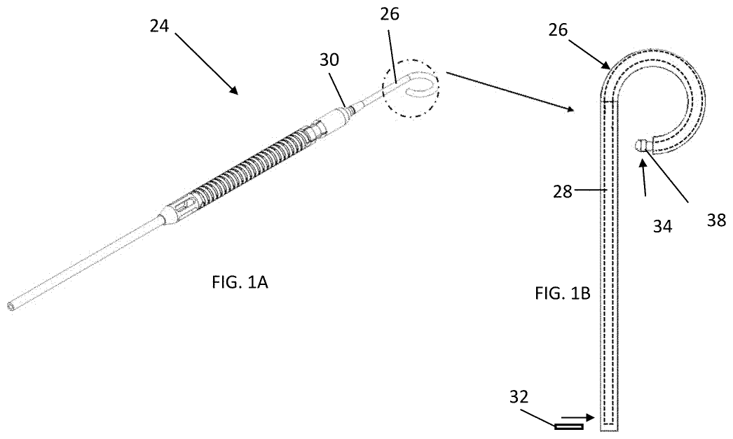

Note that while the discussion below focuses on grasper devices used in the transseptal delivery of pVAD devices, the described systems and methods can also be used for other NGB cardiac therapeutic devices. Before discussing grasper embodiments, an example of a pVAD 24 will be described with respect to Figs. lA and 1B. Details concerning the operative components of the pVAD, such as its intake and output ports, pump mechanism etc., will not be described as these are known in the art. A common feature of a pVAD device is a highly flexible pigtail element typically formed of a tubular extrusion. The pigtail element extends from the rigid distal nose 30 of the device and is designed to prevent the distal nose from causing trauma to the surrounding tissue. In a pVAD to be implanted using the graspers, a modified pigtail, referred to as an advancer element 26, is used. This element has the pigtail shape of the typical pigtail included on many pVAD devices, but it differs from the typical pigtail in multiple ways. First, the advancer element 26 includes a shape memory (e.g. nitinol) element 28 extending through the pigtail (which may be a polymeric extrusion). The nitinol element may have a uniform diameter as shown, or it may have a diameter that tapers inwardly from its proximal end to its distal end to avoid kinking near the junction between the element 28 and the distal nose 30 of the pVAD. A pin 32 (FIG. 1B) may extend laterally through the extrusion of the pigtail to the distal nose 30 of the pVAD (FIG. 1A) to secure the pigtail to the nose. The pin preferably does not extend through the nitinol element, thus leaving the nitinol element floating unsecured within the extrusion. Second, the advancer element 26 includes an engageable feature 34 at its distal tip. The engageable feature 34 of the pVAD is configured to be securely engaged by an engagement device such as a snare, grasper, or alternative engagement device, and may be formed of metal or other material suitable for withstanding engagement.

In the FIG. 1B embodiment, the engageable feature 34 comprises a tip element at the end of a smaller-diameter neck 38 extending from the lumen of the pigtail's tubular extrusion. It includes a distal face with convex curvature, and a cylindrical proximal part with a generally flat proximal face that facilitates engagement using a snare or grasper jaws. The tip element and neck may form part of an insert that is inserted into the distal end of the pigtail's lumen and secured in place using adhesive, heat treatment, a threaded engagement, and/or other means. The circumference of the part of the insert that is within the lumen may have threads or a roughened surface to enhance adhesion between the insert and the interior of the lumen.

The tip element shown is one example of a type of engageable element for the pVAD. Others might include holes, recesses or grooves that receive corresponding pins, teeth, detents, wires, etc. of the engagement device. These features may be on the advancer or on the distal nose of the pVAD. In one specific example, an annular groove is formed around the circumference around the distal nose

During delivery of the pVAD to its target location in the heart, the advancer element 26 is connected to the grasper and pulled by the grasper through the vasculature. The engageable feature/tip element 34 provides a secure member that the engagement device can securely engage for this important step in the process. After the pVAD is positioned in the heart and released from the engagement device, the shape memory element 28 within the advancer allows the pigtail (which is pulled to a straightened shape as the pVAD is drawn through the vasculature) to return to its curled configuration. Without the shape memory element 28 the polymeric extrusion of the pigtail could deform as a result of the tensile forces occurring as it is pulled through the vasculature and thus be left with a straightened shape that could scrape the endoluminal surface at the implant site.

Graspers

### First Embodiment

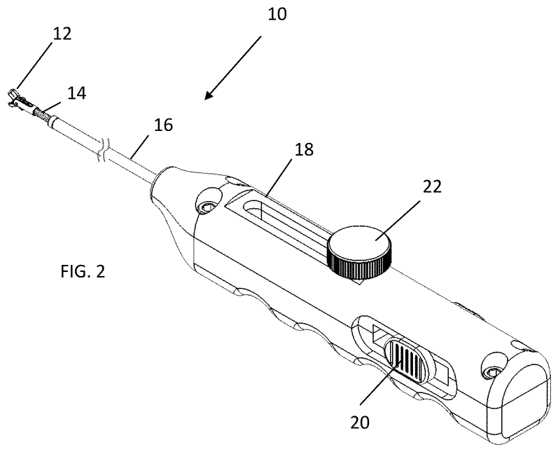

A first embodiment of a grasper 10 is shown in FIG. 2. The grasper 10 has a jaw comprised of jaw members 12 mounted on a flexible shaft 14. In preferred embodiments the shaft is a coil shaft 13 to give optimal flexibility while avoiding kinking, but alternative embodiments might make use of alternative shaft materials. An optional locking sleeve or tube 16 is longitudinally slidable over the shaft 14 between a locking position disposed over the closed jaw members 12 to prevent them from unintentionally opening, and an unlocked position in which it is disposed proximal to the jaw members so that they can be opened and closed. The locking tube or sleeve may be constructed in various ways. In one example the locking sleeve 16 is formed of a braid material. This allow the flexibility given to the shaft by the coil to be maintained, and prevents radial stretch around the jaw members, thus maintaining them in the closed position. As another example, the sleeve may be a tubular extrusion of polymeric material.

The grasper has a handle 18 equipped with a jaw actuator 20 on the handle 18 to move the jaws between opened and closed positions, and a locking tube actuator 22 used to advance the locking sleeve 16 along the shaft 14 between the locking position and the unlocked position. The handle may include lock(s) actuators engageable to lock the jaws in the closed position and/or lock the tube in the distal position, in each case to prevent unintending release of the cable or pVAD.

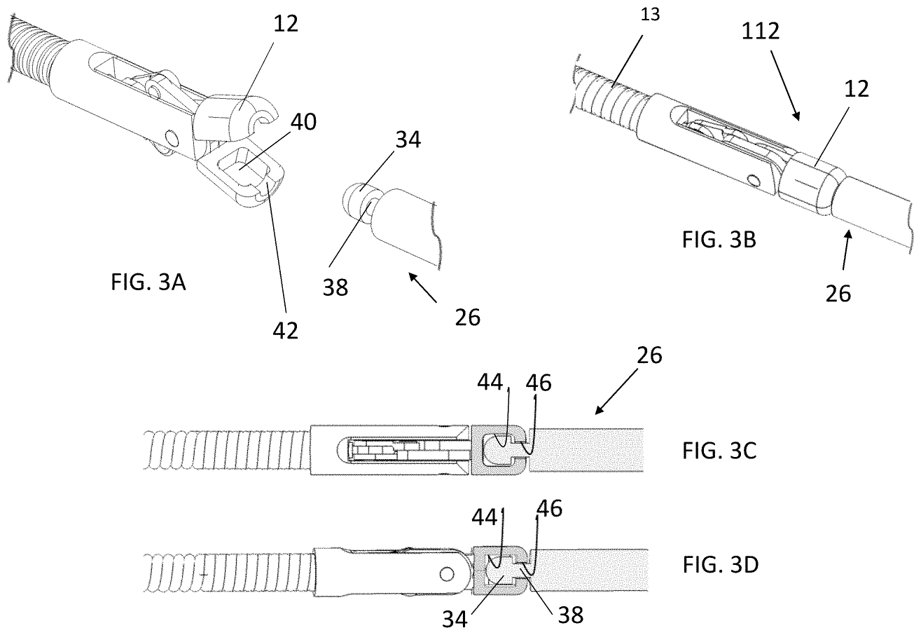

Details of the jaw members 12 of the first embodiment are shown in FIG. 3A. One or both jaw members 12 includes a seat 40 proportioned to receive the tip element 34 of the advancer 26. In the embodiment that is shown, each jaw member includes a seat 40. At the distal tip of the jaw member, a shoulder 42 extends radially inwardly. When the jaw members are moved to the closed position (FIG. 3B), the seats 40 and shoulder 42 form a chamber 44 and a through-opening 46 extending from the chamber 44 out the distal tip of the grasper (FIGS. 3C and 3D). The chamber 44 has a first diameter sized to accommodate the tip element 34 of the advancer, and the through-opening 46 has a second, smaller, diameter, sized to accommodate neck 38 of the advancer. This captures the tip element 34 within the chamber 44 when the jaws are in the closed position.

Referring again to FIG. 3B, the jaws members 12 may be shaped to help the grasper, when closed, to pass smoothly through the vasculature. To this end, the jaw members may have a shape that gives the closed jaw assembly 112 a generally cylindrical exterior wall surface when in the closed position. Moreover, the distal tip of the closed jaw assembly may have a surface that is curved in the distal direction to form a curved distal nose. The curved surface pay be a partially spherical surface, or it may possess alternative curvature.

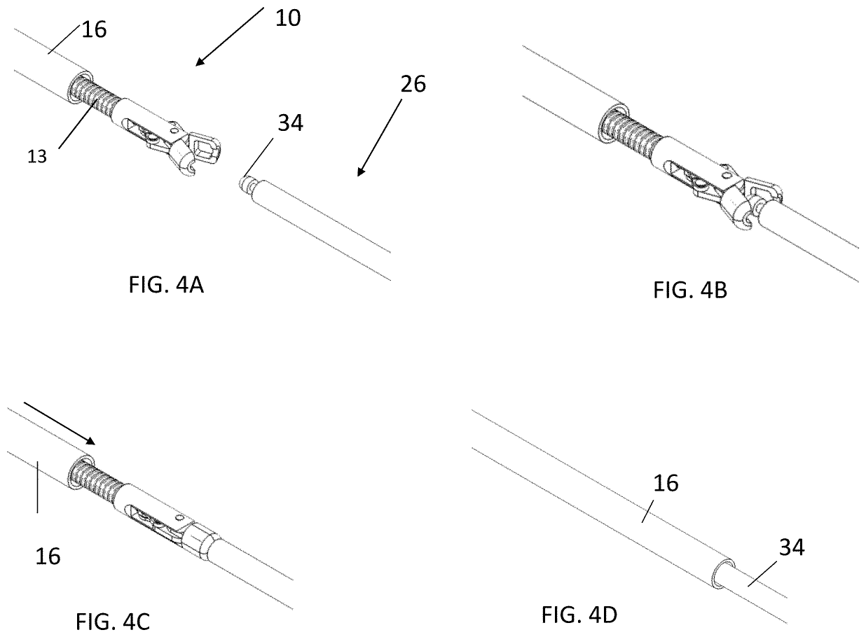

FIGS. 4A-4D illustrate the steps of securely engaging the advancer's distal tip 34 using the grasper 10. The advancer 26 is moved from its curled position to a generally straightened position (FIG. 4A) and inserted between the jaws of the grasper (FIG. 4B). The jaws are moved to the closed position (FIG. 4C) to capture the engageable feature/tip element 34 within the chamber 44 (FIGS. 3C and 3D) defined by the jaw assembly. As discussed with respect to FIGS. 3C and 3D, in this position the through opening 46 is disposed around the neck 38 of the advancer, and thus the shoulder 42 defining the through-opening 46 prevents the tip element 34 from exiting the chamber 44. When the tip element 34 is engaged by the jaw members, the advancer 26 and the jaw assembly are preferably aligned to allow smooth movement over the locking sleeve over the jaw assembly and advancer. In one embodiment, the internal walls of the chamber 44 and the circumferential wall of the tip element interact so that the jaws of the grasper and the tip element 34 remain aligned along a common longitudinal axis when the element 34 is enclosed within the chamber 34, maintaining concentricity of the jaws with the tip element 34. For example, the chamber 44 may be shaped as a negative of the tip element 34.

The locking sleeve 16 is advanced distally (FIG. 4D) to ensure that the jaws are retained in the closed position despite the significant forces pulling forces that may be applied to grasper 10 as it is used to pull the pVAD through the vasculature and across the inter-atrial septum. The locking sleeve of this and the subsequent embodiments is preferably configured to be advanced over the jaws and the full length of the advancer and into contact with the distal nose 30 (FIG. 1A) of the advancer, which may be a rigid component. This brings the grasper 10 and pVAD into longitudinal alignment. It also places the transition, along the exterior of the grasper/pVAD assembly, between the grasper 10 and the pVAD at the distal nose 30, giving the assembly a smooth exterior surface for smooth passage through the vasculature and heart. For this reason, the outer diameter of the locking sleeve 16 is preferably selected to be approximately equal to the maximum outer diameter of the distal nose 30, in other embodiments, however, the outer diameter of the locking sleeve 16 may be smaller than the maximum outer diameter of the distal nose 30.

### First Embodiment—Handle Features

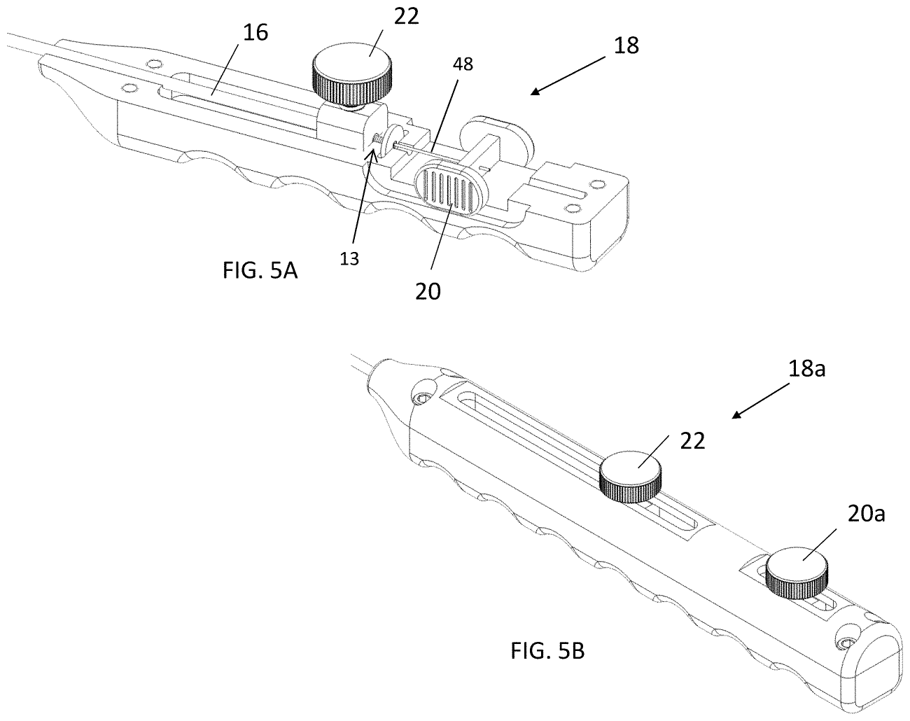

FIG. 5A shows the handle 18 of FIG. 2 with a portion of the housing removed. As shown, in this embodiment the jaw actuator 20 is in the form of a slidable member that may be moved in proximal and distal directions relative to the handle 18 to adjust the tension on a pullwire 48 to cause opening/closing of the jaws. This embodiment is configured so that activation of the pullwire causes simultaneous movement of both jaw members, although in alternative embodiments one jaw is fixed and the other is actively moveable by the pullwire. Note that the term “pullwire” or “wire” is not intended to mean that the pullwires must be formed of wire, as that term is used more broadly in this application to represent any sort of tendon, cable, or other elongate element the tension on which may be adjusted to cause opening/closing of the jaws. In the preferred embodiment, however, the pullwire is a solid wire running through the lumen of the coil shaft 13 which, as shown, extends into the handle. Use of the solid wire helps prevent stretching of the grasping catheter when it is used to deliver the pVAD to a target site during use. The actuator 20 may optionally be lockable in the opened or closed position.

The handle may be configured for user-actuated movement of the jaws from the open position to the closed position, and from the closed position to the open position, or springs may be used to return the jaws to the open or closed position when the user releases or unlocks the actuator 20.

In this embodiment, the locking tube actuator 22 is the illustrated thumb screw member that can be moved in a distal direction to advance the locking sleeve 16 along the shaft 14 to retain the jaws in the closed position, and then optionally tightened to retain the locking sleeve 16 in the distal/closed position. When the thumb screw is unlocked, movement of the thumb screw in a distal direction relative to the handle 18 withdraws the locking sleeve 16 from the jaws, allowing the jaws to be opened. A spring element may be used to bias the locking tube in the proximal or distal position, so that, for example, once the thumbscrew is unlocked the locking sleeve 16 moves proximally under action of this spring's spring force (or vice versa).

FIG. 5B shows a modified handle 18a which differs from the FIG. 5A handle in that the pullwire actuator 20a is a locking thumb screw mechanism of the type described for the locking tube actuator. This thumb screw mechanism can be moved in distal and proximal directions relative to the handle to open and close the jaws, and then tightened to retain the jaws in the open/closed position. As with the FIG. 5A embodiment, the handle 18a may be configured for user-actuated movement of the jaws from the open position to the closed position, and from the closed position to the open position, or springs may be used to return the jaws to the open or closed position when the user releases or unlocks the actuator 20a.

Note that the features of the handle described here are not intended to limit the scope of the claims, as handle and actuator configurations suitable for adjusting tension on a pullwire, moving a tube in proximal and distal directions, and locking such mechanisms are well understood by those skilled in the art.

Additional embodiments of graspers will now be described. In these descriptions and their corresponding figures, reference numbers used in connection with the first embodiment will be used to denote similar features present in the additional embodiments. It should also be understood that features of the various embodiments may be combined with features of the other embodiments to produce additional embodiments without deviating from the scope of this application. Moreover, handle features of the type described with respect to the first embodiment, or suitable alternatives, may be used in each of the subsequent embodiments.

### Second Embodiment

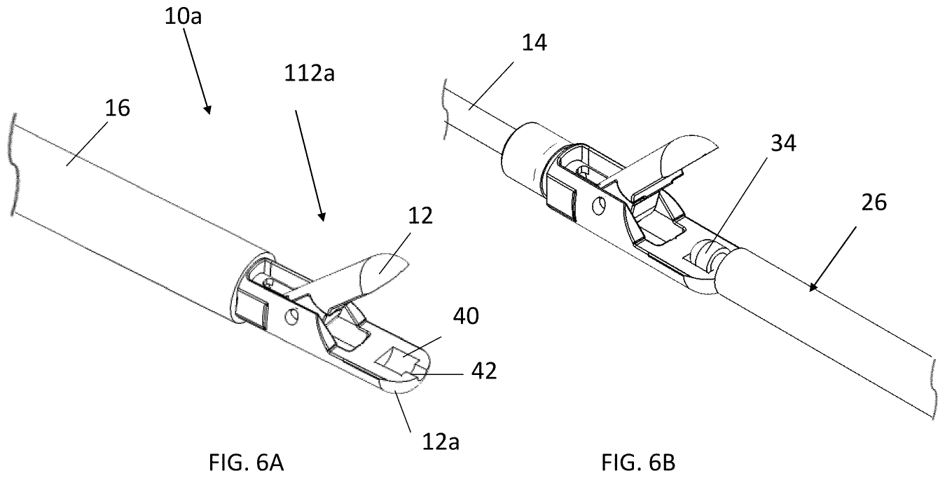

FIGS. 6A and 6B show a second embodiment of a grasper 10a, which differs from the first embodiment in that the jaw assembly 112a of the grasper 10a includes a moveable jaw member 12 like the jaw members described in connection with the first embodiment, and a fixed jaw member 12a. Each of the jaw members 12, 12a preferably has the shape and capture features discussed in connection with the first embodiment, to facilitate secure engagement of the advancement by the jaws and to allow smooth passage of the closed jaws through the vasculature. As with the first embodiment, closing the jaw member to engage the grasper and the advancer preferably concentrically aligns the advancer and jaw assembly and allows smooth transfer of a locking tube or sleeve 16 over the jaw assembly and advancer to prevent inadvertent movement of the jaws out of the closed position.

### Third Embodiment

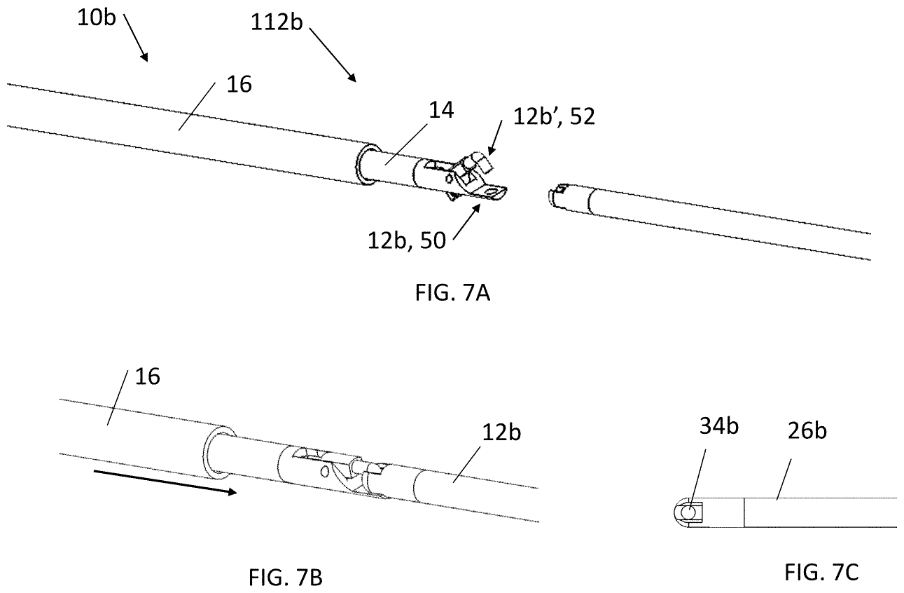

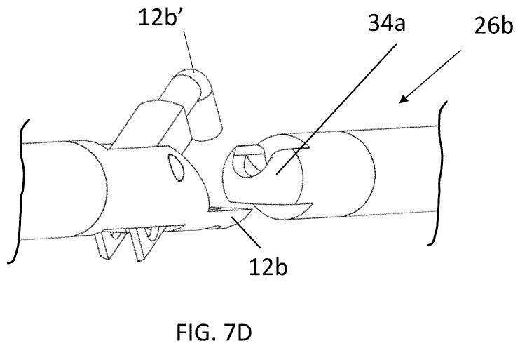

In a third embodiment of a grasper 10b, depicted in FIG. 7A, the jaw assembly 112b includes a fixed jaw member 12b having a through hole 50, and a moveable jaw member 12b′ having a pin 52 shaped such that it passes into the through hole 50 when the jaw member 12b′ is moved from the open position shown in FIG. 7A to the closed position shown in FIG. 7B. The moveable jaw member may be pivotable as shown, or it may move in parallel to the fixed jaw member.

This embodiment is designed to engage an advancer 26b having as its engageable feature a through hole 34b, so that when the advancer 26b is inserted into the jaw assembly 112b and the jaw member 12b′ is moved to the closed position, the pin 52 passes through the through hole 34b in the advancer and through the through hole 50 in the jaw member 12b.

A locking tube or sleeve 16 is optionally operable in similar fashion to the locking tube of the first embodiment through third embodiments to prevent inadvertent movement of the jaws out of the closed position. In this and the other embodiments that use a locking sleeve, the locking sleeve may have a braid construction as discussed in connection with the first embodiment, or it may have an alternative construction.

While the fixed jaw member 12b, the pivotable jaw member 12b′ and the advancer 26b can be designed to have a variety of different shapes, in the present embodiment the through hole 50 extends through a distal tip element 34a of the advancer. The distal tip element 34a includes a first planar surface positioned to sit flush against a corresponding planar face of the fixed jaw member 12b, as shown in FIG. 7D. The opposite side of the tip element 34a has a trough that the moveable jaw member seats in when it is moved to the closed position engaging with the through hole 50. Because features of the advancer 26b mate with the tip element 34a in this way, the advancer and grasper are concentrically and linearly aligned about a common longitudinal axis, allowing the locking sleeve to smoothly slide over the advancer and grasper assembly and maintain them in alignment for smooth delivery to the target location.

### Fourth Embodiment

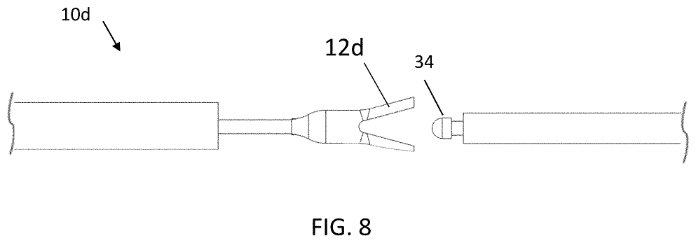

A fourth embodiment of a grasper 10c is shown in FIG. 8. In this embodiment, the grasper includes a jaw 12d biased in an opened position. locking tube or sleeve 16 operates to move the jaws to the closed position, thus eliminating the need for a pull wire. Each of the jaw members 12d preferably has the shape and capture features discussed in connection with the first embodiment, to facilitate secure engagement of the advancement by the jaws and to allow smooth passage of the closed jaws through the vasculature. In use, the advancer tip 34 is positioned between the jaw members, and the locking sleeve is advanced over the jaws and advancer, closing the jaws and retaining them in the closed position. To separate the grasper from the advancer, the sleeve is withdrawn. Once the sleeve uncovers the jaws, the jaws spring to the open position, thus releasing the advancer.

### Fifth Embodiment

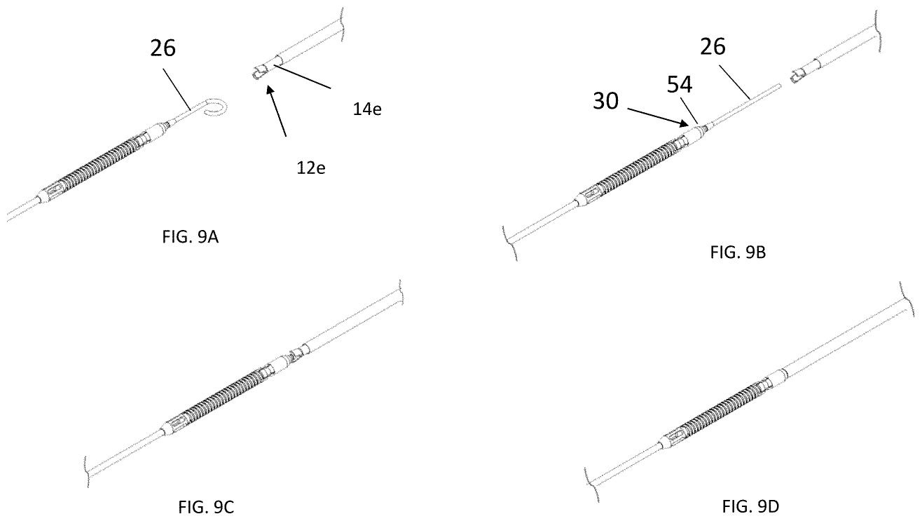

In a fifth embodiment illustrated in the sequence of FIGS. 9A-9D, the jaws 12e are mounted on a shaft 14e that includes a lumen. As with the fourth embodiment, the jaws are spring biased in the open position.

To lock the grasper to the pVAD, the advancer 26 is inserted between the jaws of the jaw member 12e and into the lumen of the shaft 14e (FIGS. 9B-9C). Once the advancer 26 is fully inserted into the lumen, the locking tube or sleeve 16 is advanced distally (FIG. 9D) to squeeze the jaws closed. In this step engagement elements of the jaw capture the corresponding engageable feature of the advancer. As an alternative the engagement features (e.g. tip 34) discussed above, the pVAD shown in this embodiment includes one in the form of an annular groove 54 or an annual ridge or shoulder at the distal nose 30 of the pVAD (i.e. proximal to the advancer 26). Features (e.g. detents) in the jaws engage, or seat in, the groove as they close on the advancer. A lock positioned at the handle (not shown) of the grasper locks the outer tube in its distal position until the user is ready to release the pVAD from the grasper, at which time the user unlocks the outer tube and withdraws it from its distal position over the jaws. In a modified version of the fifth embodiment, the jaws may be closed by pullwire actuation as described above, and the locking tube or sleeve 16 may be optionally eliminated.

### Sixth Embodiment

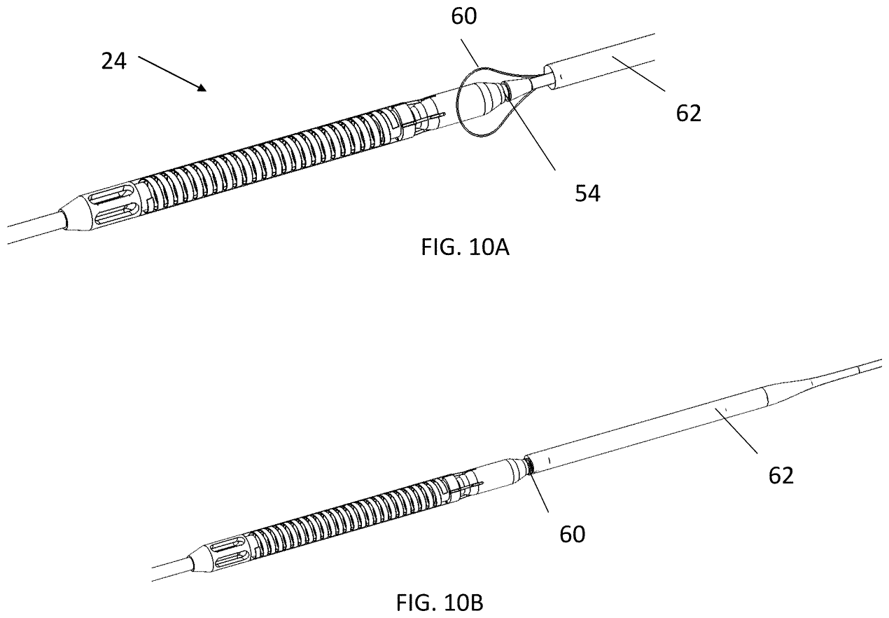

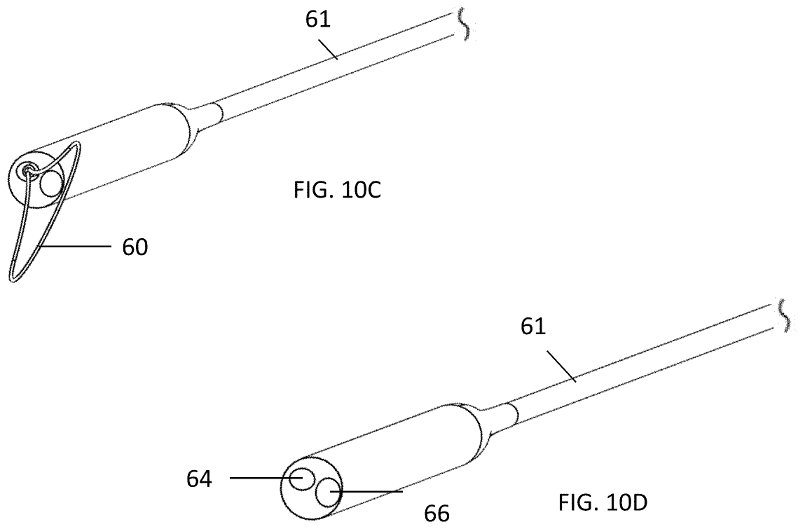

In a sixth embodiment, a snare, rather than a jaw assembly, is used to engage the pVAD. Referring to FIGS. 10A and 10B, the sixth embodiment is used to securely engage a pVAD by engaging engagement features on the pVAD using a snare loop 60 disposed at the end of a snare shaft. The snare and snare shaft can be configured in a number of different ways, but in the embodiment shown in FIGS. 10C and 10D, the snare loop extends from a flexible snare catheter 61 that includes two lumens: a snare lumen 64 and an advancer lumen 66. In use, the snare 60 is positioned with its shaft extending through the snare lumen 64 as shown in FIG. 10C. The advancer 26 is passed through the snare loop 60 and inserted into the advancer lumen 66 as shown in FIG. 10A. The engagement features such as the groove, ridge or shoulder 54 on the pVAD's distal nose described above in connection with the fifth embodiment may be used for this purpose. A snare tensioner 62, which may be an elongate tube, is advanced over the snare for use in tightening the snare to the closed position shown in FIG. 10B. To separate the pVAD from the grasper, the snare tensioner 62 is withdrawn, the snare is loosened, and the snare and snare shaft are pulled off of the advancer.

### Seventh Embodiment

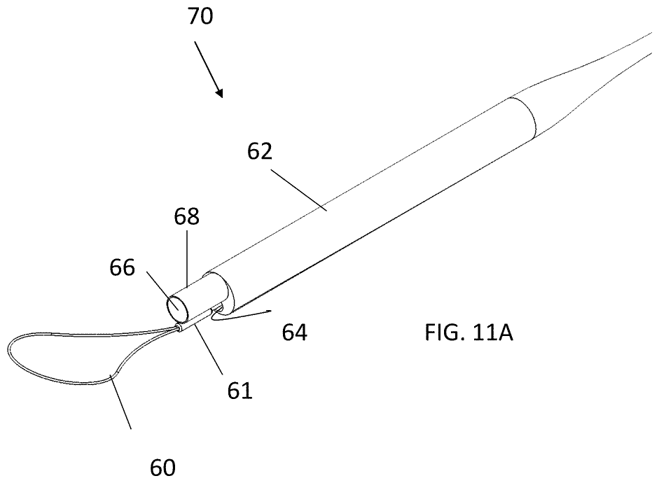

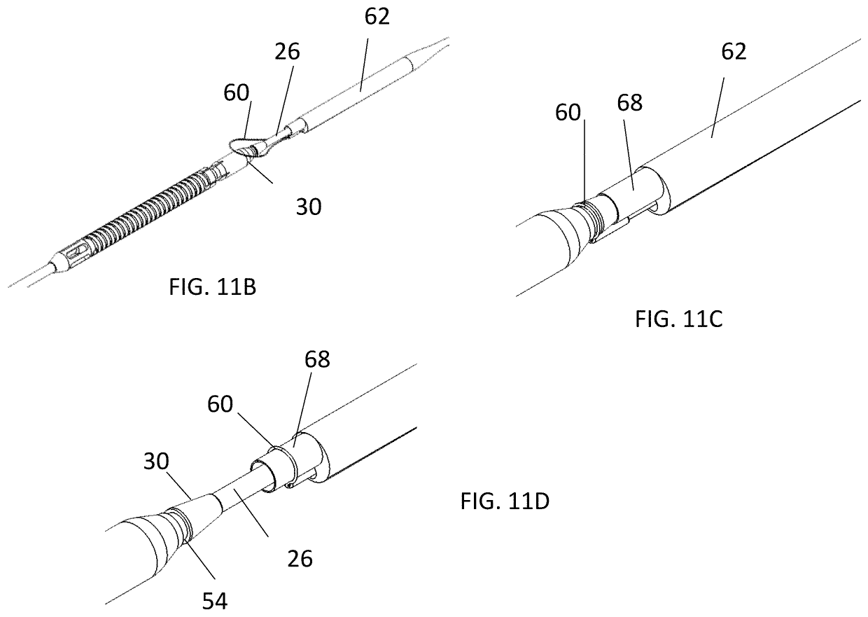

In a seventh embodiment shown in FIGS. 11A-11D, a grasper assembly 70 includes a snare tensioner 62 which has a snare lumen 64, an advancer lumen 66 which extends into the tensioner 62 via an extension 68 extending distally from the tensioner. In use, the snare 60 is positioned with its shaft extending through the snare lumen 64 as shown in FIG. 11A. In this initial configuration, the snare loop 60 may be positioned over the extension 68 as described in connection with FIG. 11D.

The advancer 26 of the pVAD is inserted into the pVAD lumen as shown in FIG. 11B, and the snare is advanced over the advancer onto the pVAD. If the snare loop is on the extension, this latter step entails sliding the loop off the extension 68 and onto the advancer. The snare is then tightened by pushing the advancer 26 distally while maintaining tension on the shaft of the snare, advancing the extension 68 towards or onto the pVAD distal nose 30 and consequently tightening the snare onto an engagement feature on the pVAD. See FIG. 11C.

To release the pVAD from the snare, the loop of the snare is withdrawn proximally from the pVAD onto the extension 68 as shown in FIG. 11D, and the grasper assembly 70 is withdrawn from the pVAD.

### Eighth Embodiment

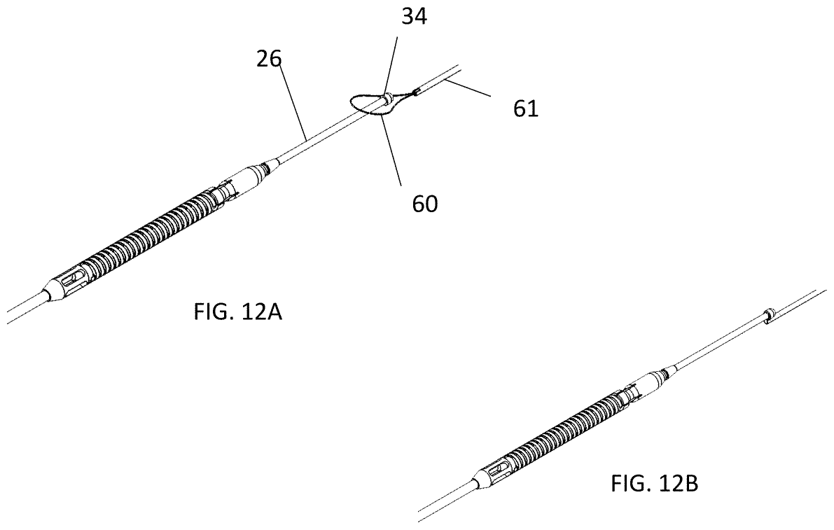

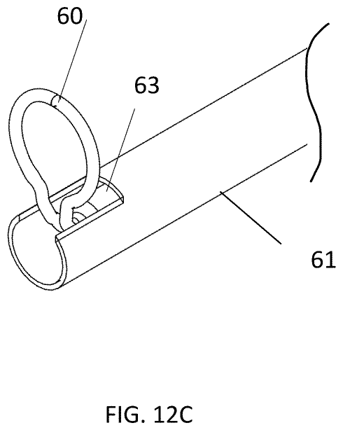

In an eighth embodiment, depicted in FIGS. 12A through 12C, a snare loop 60 extends from the distal end of a snare catheter 61. The catheter 61 is a flexible tubular device having a notch 63 in its sidewall at the distal tip, and with the snare extending laterally from the notch. In use the snare 60 is used to engage a feature on the advancer 26, such as the tip element 34. The snare is tightened onto the advancer. Because the snare extends laterally from the notch, tightening the snare draws the advancer snugly against the side of the snare catheter 61 in order to bring the longitudinal axis of the advancer and the longitudinal axis of the snare catheter close together.

### Ninth Embodiment

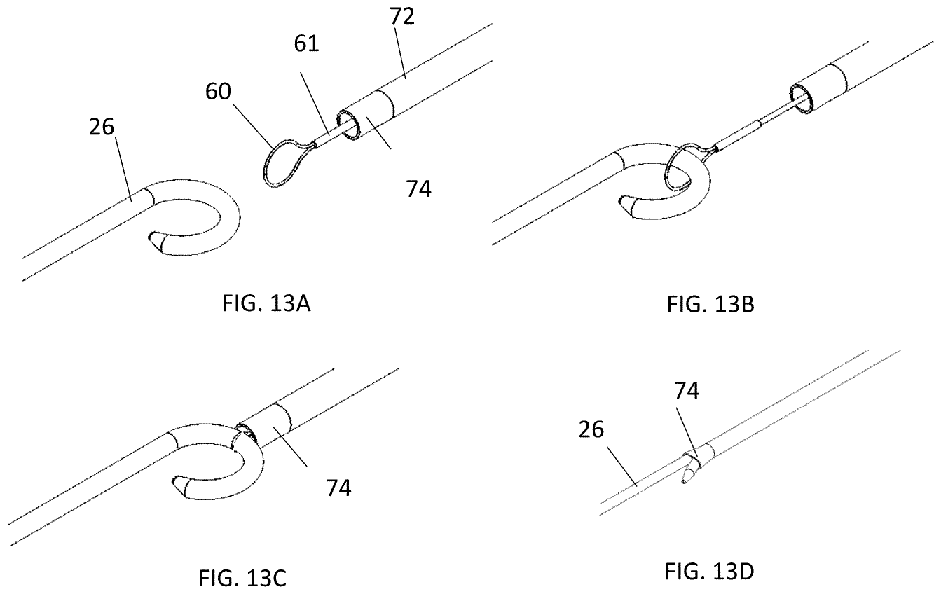

In a ninth embodiment, shown in FIGS. 13A-13D, the snare 60 is extendable from a flexible snare catheter 72 having a deformable distal tip 74. For example, the distal tip 74 may be formed of a material that is much softer than the material of the snare catheter 72. The snare loop 60 is positioned over the advancer and then tightened. FIGS. 13B, 13C. Afterwards, the snare loop 60 is withdrawn by pulling on the snare shaft 61. This action draws the advancer into the distal tip 74 of the snare catheter, causing the advancer 26 to fold on itself and to deform the distal tip 74 as shown in FIG. 13D. In this embodiment, the nitinol element of the advancer described in connection with FIG. 1 may be excluded from the advancer 26 so the advancer is sufficiently flexible to fold and enter the snare catheter.

### Tenth Embodiment

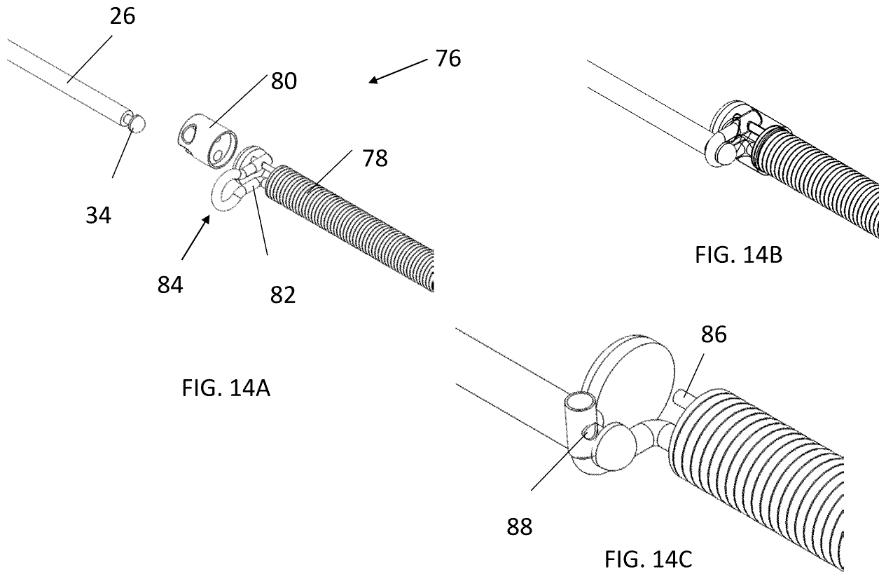

In a tenth embodiment of a grasper 76 shown in FIGS. 14A-14C, the grasper includes a shaft 78, which may have a coil construction as shown. A cap 80 is positioned on the distal end of the shaft 78. In some embodiments, the cap may extend proximally over the length of the shaft 78 instead of being positioned only at the distal end as shown.

A quick release snare 82 ends through the shaft 78 and includes a distal end that can be positioned to exit and then reenter the cap 80 through holes in the cap's sidewall, thus forming a loop 84. This snare may optionally be a nitinol wire having a cylindrical cap or tube (e.g. formed of steel) having a lateral hole 88 through its cylindrical sidewall. A wire or pin 86 extends through, and is longitudinally moveable with respect to, the catheter 72. The wire 86 is oriented so that it may be positioned through the hole 88 in the snare's cap to retain the snare in the looped position as shown in FIGS. 14A and 14B. An actuator mechanism in the handle (not shown) at the proximal end of the shaft allows the user to move the wire from the distal position shown in FIGS. 14A and 14B to the withdrawn position shown in FIGS. 14C.

To engage an advancer using the grasper 76, the snare is placed in the looped position around the element 34 of advancer 26 and secured in the loop position using the wire 86. To separate the advancer from the grasper 76, the user withdraws the wire 86 using the handle, causing the wire 86 to withdraw from the hole 88. Once the wire 86 is withdrawn, the loop 84 opens up, releasing the tip element 34 of the advancer 26.

### Eleventh Embodiment

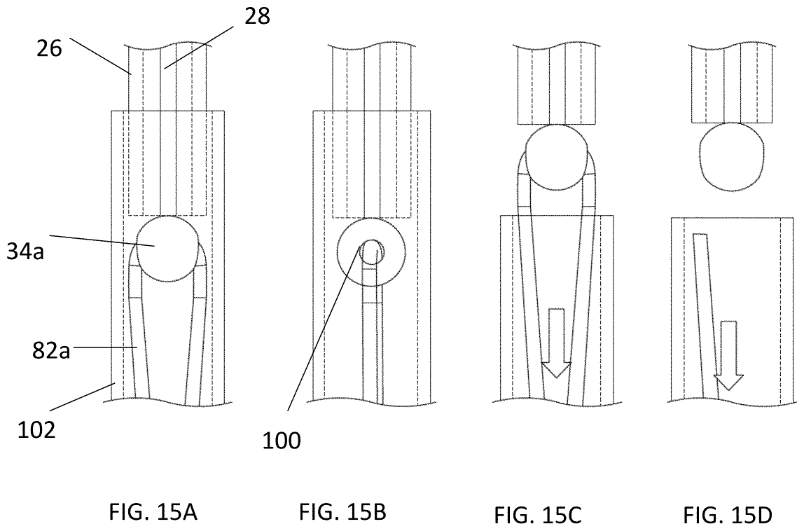

An eleventh embodiment of a grasper assembly is like the tenth embodiment in that it uses a wire or snare 82a that has a first position in which it is formed into a loop to engage a portion of the pVAD, and a second position in which it is released from the looped position in order to disengage the pVAD. In this particular example, when in the first position the loop is formed with the snare extending through a part of the advancer 26. FIGS. 15A-15D show that the engagement feature 34a on the advancer may be a tip element 34a having a hole 100 extending through it. As one example, the nitinol element 28 that extends through the advancer may have a ball tip attached to it as shown. The snare 82a, which may be a loop of suture or wire, is inserted through the hole 100 to form the loop, and an outer sheath 102 is passed over the advancer 26 while tension is maintained on the loop, locking the pVAD to the grasper assembly (FIGS. 15A-15B). To release the pVAD from the grasper assembly, the sheath 102 is retracted to expose the suture and ball. FIG. 15C. Finally, the snare is retracted from the ball 34a to release the advancer from the grasper assembly. Retracting of the snare may be performed by releasing an end of the suture or wire forming the snare, or by cutting the snare.

### Twelfth Embodiment

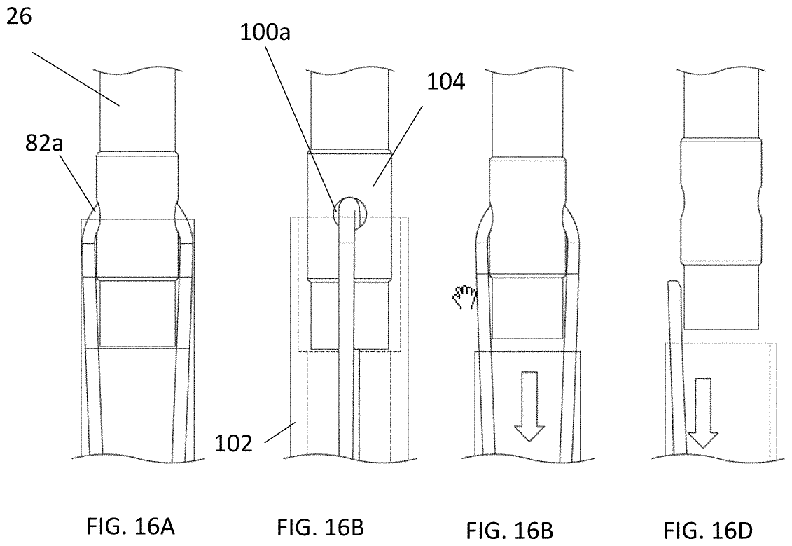

A twelfth embodiment, shown in FIGS. 16A-16D, is similar to the eleventh embodiment except that in lieu of the ball 34a on the advancer, the advancer used in the twelfth embodiment includes a hole 100a that receives the snare 82a. The hole may be on a portion of the advancer reinforced using a collar 104 as shown.

### Thirteenth Embodiment

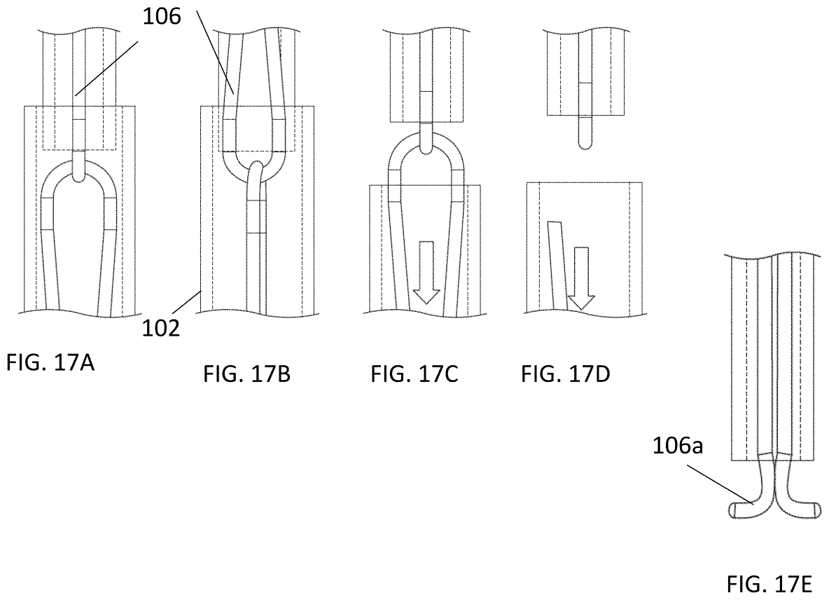

A thirteenth embodiment, shown in FIGS. 17A-17D, is similar to the eleventh embodiment except that in lieu of the ball 34a on a nitinol core of the advancer, the advancer used in the thirteenth embodiment includes a nitinol shape set loop 106. The nitinol loop may include a distal part that extends beyond the distal end of the outer jacket of the advancer as shown. To attach this grasper assembly to the advancer, the suture or wire forming the snare loop 82a is threaded through the nitinol loop 106, and the distal end of the advancer is inserted into the outer sheath 102. To release the pVAD from the grasper assembly, the sheath 102 is retracted to expose the snare and nitinol loop. FIG. 17C. Then, the snare is retracted from the nitinol loop to release the advancer from the grasper assembly.

Note that while the nitinol element 106 of the advancer is shown formed into a loop, this element may have other shapes that may be engaged by the suture/snare. For example, in FIG. 17E an alternative nitinol element 106a is shown, which is shaped in an anchor type of configuration to provide a ledge for the snare loop to engage.

### Fourteenth Embodiment

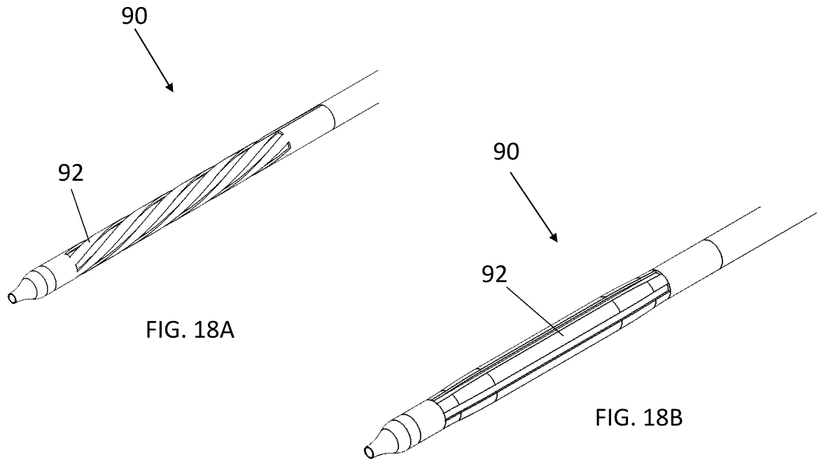

FIGS. 18A and 18B show a grasper 90 configured for insertion into a lumen extending through a pVAD or pVAD advancer. Grasper 90 includes an expandable collet formed of a plurality of struts 92 arranged in a first, compressed position. In FIG. 18A the compressed position is shown as one in which the struts wrap helically relative to the longitudinal axis of the grasper 90. The struts are moveable to a second, expanded, position in which the struts extend longitudinally and bow outwardly relative to the longitudinal axis of the grasper 90. To engage a pVAD, the collet is positioned in the compressed position and inserted into the lumen of a pVAD or pVAD advancer, and then the collet is moved to the expanded position to expand the collet within the lumen. The collet expands into secure contact with the surrounding walls of the pVAD/advancer lumen, allowing use of the grasper 90 to pull the pVAD through the vasculature without separation. Once the pVAD is in the desired position, the collet is returned to the compressed position and then withdrawn from the lumen of the pVAD/advancer.

### Fifteenth Embodiment

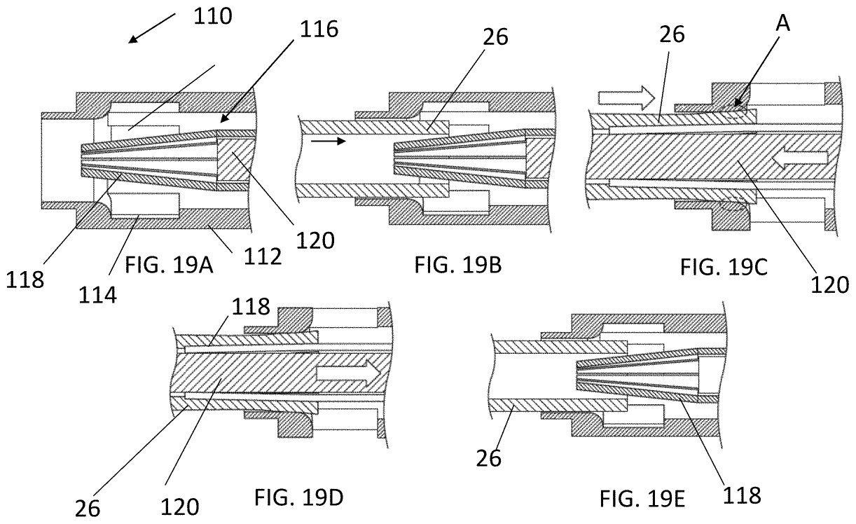

An alternate embodiment using an expandable collet is shown in FIG. 19A. In this embodiment, the grasper assembly 110 includes an outer tubular member 112, the distal end of which is shown in the drawings. The outer member 112 has viewing slots or transparent regions 114 near its distal end. An inner member 116 having a lumen is longitudinally moveable within the outer member 112. The inner member 116 has on its distal end an expandable collet 118. An inner member 120 extends through the lumen of the inner member 114. The inner member 120 is a wire that is tapered so that its lateral diameter increases in the distal to proximal direction.

The grasper assembly 110 is designed for use with an advancer 26 having a hollow lumen and open distal end. In use, the advancer 26 is inserted into the lumen of the outer member from the distal end of the outer member as shown in FIG. 19B, so that it is positioned over the distal tip of the collet 118. The viewing slots 114 allow the user to confirm that the distal end of the advancer has been advanced over the collet. Next, the inner member 120 is advanced within the collet in the distal direction, causing outward expansion of the collet within the advancer 26. See FIG. 19C. Expansion of the collet expands the advancer against the inner circumference of the outer member and in doing so creates a circumferential mechanical lock of the advancer 26 between the outer member 112 and the collet 118 in circumferential region A marked in FIG. 19C. During this step, the advancer may continue to be pushed into the lumen to ensure engagement. Once it is time to release the pVAD from the grasper, the inner member 120 is retracted as shown in FIG. 19D, causing the collet to return to the compressed position shown in FIG. 19E, releasing the lock on the advancer 26 and allowing removal of the advancer from the outer member.

### Sixteenth Embodiment

As with some of the embodiments described above, the sixteenth embodiment is designed to be used with a polymeric advancer 26 having an open lumen at its distal end. However, in this embodiment, the lumen is modified to include a ring or disk 126 embedded within the lumen. The disk has a circumferential exterior surface that is knurled or provided with some other type of surface roughness that will enhance engagement between the exterior surface of the disk and the interior surface of the advancer 26 as the advancer 26 is formed using a reflow technique. The disk includes a distal face 128 having an opening 130.

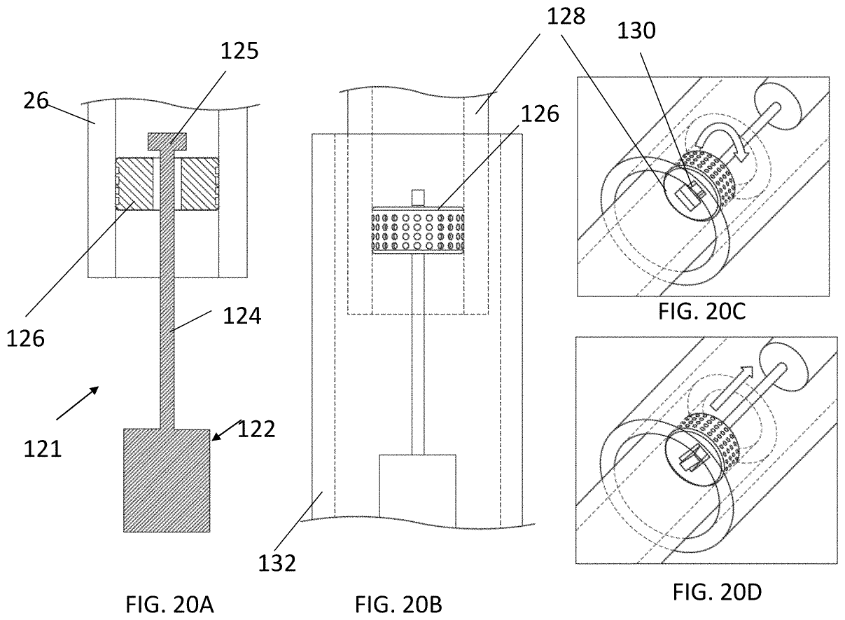

In accordance with the sixteenth embodiment shown in FIGS. 20A-20D, a grasper assembly 121 includes an outer tubular member 132, and shaft 122 provided with an insertion portion 124 insertable into the lumen of the advancer 26, and a tip member 125. The tip member 125 and opening 130 are shaped so that when the shaft 122 is in a first rotational orientation with respect to the disk 126, the tip member 125 can pass through the opening 125. In the illustrated configuration, the opening 130 is a rectangular opening and the tip member 125 has a rectangular cross-section, although other shapes can be used.

To use the grasper assembly, the tip member 125 is inserted into the lumen of the advancer 26 and through the opening 130 of the disk 126. The shaft 122 is then rotated to orient the tip so that it cannot pass back through the opening 130 unless rotated back into alignment with the opening. Next, the outer member 132 is advanced over the shaft 122 and the distal part of the advancer 26 in order to capture the advancer between the outer member 132 and the disk 126, thus engaging the grasper assembly 121 to the pVAD advancer. Once the pVAD is positioned as desired, the shaft 122 is rotated about its longitudinal axis as indicated by the arrow in FIG. 20C, rotating the tip from the position shown in FIG. 20C to the position shown in FIG. 20D. This orients the tip 125 of the shaft 122 in alignment with the opening of the disk 130, allowing the shaft 122 to be withdrawn as indicated by the arrow in FIG. 20D. The outer member is 132 is then withdrawn from the advancer.

1. A system for use in transseptally delivering a cardiac therapeutic device (“CTD”) to a target site in the heart, comprising:
a CTD having an elongate flexible advancer on its distal end;
a flexible member proportioned for introduction into a vasculature and having a length to extend from the right subclavian vein, through the heart via a transseptal puncture, and to a femoral artery such that a first end of the flexible member is external to the patient at the right subclavian vein or other venous access site and a second end of the flexible member is external to the patient at the femoral artery, the flexible member having a grasper having a jaw assembly moveable to a closed position to releasably engage with the advancer.

2. The system of claim 1, wherein the advancer has a tip element and the jaw assembly includes at least one jaw member moveable between an open position for receiving the tip element and a closed position in which the jaw member is releasably engaged with the tip element.

3. The system of claim 1, further including a sleeve slidable over the jaw assembly and at least a portion of the advancer when the jaw member is in the closed position.

4. The system of claim 3, wherein the sleeve is formed of a braid material.

5. The system of claim 2, wherein the jaw assembly includes a pair of jaw members, at least one of the jaw members defining a seat proportioned to receive the tip element of the advancer.

6. The system of claim 2, wherein the at least one jaw member is positioned on a coil shaft.

7. The system of claim 6, wherein the flexible member includes a pullwire extending through the coil shaft to a handle, the handle having an actuator moveable by a user to adjust tension on the pullwire to move the jaw member between opened and closed positions.

8. The system of claim 7, wherein the grasper includes a pair of jaw members, and movement of the actuator simultaneously moves the pair of jaw members between opened and closed positions.

9. The system of claim 5, wherein the seat is shaped as a negative of the tip element.

10. The system of claim 9, wherein the seat is defined by each of the two jaw members.

11. The system of claim 1, wherein when the jaw assembly is releasably engaged with the advancer, the jaw assembly and advancer are concentrically aligned.

12. The system of claim 3, wherein when the jaw assembly is engaged with the advancer, the locking sleeve is advanceable over the jaw member and advancer and into contact with the distal end of the pVAD or other cardiac therapeutic device.

13. The system of claim 2, wherein the jaw member includes a pin and wherein the advancer includes a through hole positioned to receive the pin when the jaw member is moved the closed position.

14. The system of claim 13, wherein the jaw assembly further includes a second, fixed, jaw member.

15. The system of claim 3 wherein the jaw member is spring biased in an open position, and wherein the sleeve is advanceable over the jaw member to move the jaw member from the open position to the closed position.

16. The system of claim 1, wherein the CTD includes a cannula and the advancer extends from the distal end of the cannula, and wherein the flexible member includes a lumen and a jaw member, the advancer extendable into the lumen, the jaw member moveable from an open position for receiving a portion of the cannula and a closed position in which the jaw member is releasably engaged with the cannula.

17. The system of claim 16 wherein the jaw member is spring biased in an open position, and wherein the system further includes a sleeve slidable over the jaw member and at least a portion of the advancer, the sleeve is advanceable over the jaw member to move the jaw member from the open position to the closed position.

18. A system for use in transseptally delivering a cardiac therapeutic device (“CTD”) to a target site in the heart, comprising:
a CTD having an elongate flexible advancer on its distal end;
a flexible member proportioned for introduction into a vasculature and having a length to extend from the right subclavian vein, through the heart via a transseptal puncture, and to a femoral artery such that a first end of the flexible member is external to the patient at the right subclavian vein or other venous access site and a second end of the flexible member is external to the patient at the femoral artery, the flexible member having a snare releasably engageable with the advancer.

19. The system of claim 17, wherein the CTD includes a cannula and the advancer extends from the distal end of the cannula, and wherein the flexible member includes a lumen and a snare, the advancer extendable into the lumen, the snare positionable around the cannula, wherein the system further includes a sleeve slidable over the snare to secure the snare on the cannula.

20. The system of claim 17, wherein the snare is positionable around the cannula, wherein the system further includes a sleeve slidable over the snare to secure the snare on the cannula.

21. The system of claim 17, wherein the flexible member further includes a lumen, wherein the snare extends from the lumen, and wherein retraction of the snare into the lumen draws the advancer into the lumen.

22. The system of claim 20, wherein the flexible member includes a deformable distal tip, wherein drawing the advancer into the lumen deforms the distal tip to enlarge the lumen at the distal tip.

23. The system of claim 17, wherein the snare includes a lateral hole and the flexible member includes a wire removably positioned in the lateral hole to engage the snare in a loop configuration, the wire retractable using a handle to release the snare from the loop configuration to release the advancer from the flexible member.

23-31. (canceled)
